(19) United States
(12) Patent Application Publication
(10) Pub. No.: US 2022/0133218 A1
(43) Pub. Date: May 5, 2022

(54) SMART JOINT MONITOR FOR BLEEDING DISORDER PATIENTS

(71) Applicant: International Business Machines Corporation, Armonk, NY (US)

(72) Inventors: Tathagato Bose, Kolkata (IN); Sourav Bhattacharjee, Durgapur (IN); Anjali Kulkarni, Bangalore (IN)

(21) Appl. No.: 17/084,710

(22) Filed: Oct. 30, 2020

(51) Int. Cl. A61B 5/00; G16H 40/67; G16H 50/20; G16H 50/30; G16H 70/60; G16H 50/70; G16H 20/30; A61B 5/107; A61B 7/00; A61B 5/11; A61B 5/01; A61B 5/02; A61B 5/103

(52) U.S. Cl. CPC A61B 5/4528; A61B 2562/028; G16H 50/20; G16H 50/30; G16H 70/60; G16H 50/70; G16H 20/30; A61B 5/6804; A61B 5/1071; A61B 7/006; A61B 5/7203; A61B 5/1121; A61B 5/1116; A61B 5/01; A61B 5/4878; A61B 5/02042; A61B 5/4023; A61B 5/1036; A61B 2562/0271; A61B 2562/0261; G16H 40/67

(57) ABSTRACT

In an approach to smart joint monitoring for bleeding disorder patients, one or more sets of data are received from a smart joint monitor, where the smart joint monitor includes one or more sensors. One or more criticalities are detected, where the one or more criticalities are detected by an artificial intelligence engine based on the one or more sets of data and a global knowledge base. One or more suggestions are determined, where the one or more suggestions are determined by the artificial intelligence engine based on the one or more criticalities and the global knowledge base.

### BACKGROUND

The present invention relates generally to the field of wearable medical devices, and more particularly to smart joint monitoring for bleeding disorder patients.

Skeletal joints are the areas where two or more bones meet. Most skeletal joints are mobile, allowing the bones to move. Skeletal joints consist of a number of different tissues. Cartilage is a type of tissue that covers the surface of a bone at a joint and helps reduce the friction of movement within a joint. The synovial membrane, which lines the joint and seals it into a joint capsule, secretes a clear, sticky fluid (synovial fluid) around the joint to lubricate it. Ligaments are tough, elastic bands of connective tissue that surround the joint to give support and limit the joint's movement. Ligaments connect bones together. Tendons are another type of tough connective tissue that attach to muscles that control movement of the joint. Tendons connect muscles to bones. Bursas are fluid-filled sacs between bones, ligaments, or other nearby structures. They help cushion the friction in a joint. The meniscus is a curved piece of cartilage in the knees and other joints.

Hemophilia is an inherited bleeding disorder in which the blood does not clot properly. This can lead to spontaneous bleeding as well as bleeding following injuries or surgery. Blood contains many proteins called clotting factors that can help to stop bleeding. The severity of hemophilia that a person has is determined by the amount of factor in the blood. The lower the amount of the factor, the more likely it is that bleeding will occur which can lead to serious health problems.

Hemophilia is caused by a mutation or change in one of the genes that provides instructions for making the clotting factor proteins needed to form a blood clot. This change or mutation can prevent the clotting protein from working properly, or it may be missing altogether. There are several different types of hemophilia. The two most common types are Hemophilia A, caused by a lack or decrease of clotting factor VIII, and Hemophilia B, caused by a lack or decrease of clotting factor IX.

### SUMMARY

Embodiments of the present invention disclose a method, a computer program product, and a system for smart joint monitoring for bleeding disorder patients. In one embodiment, one or more sets of data are received from a smart joint monitor, where the smart joint monitor includes one or more sensors. One or more criticalities are detected, where the one or more criticalities are detected by an artificial intelligence engine based on the one or more sets of data and a global knowledge base. One or more suggestions are determined, where the one or more suggestions are determined by the artificial intelligence engine based on the one or more criticalities and the global knowledge base.

Embodiments of the present invention disclose a method, and a computer program for smart joint monitoring for bleeding disorder patients. In one embodiment, one or more sets of data are collected from a wearable device, wherein the wearable device is a smart joint monitor that includes one or more sensors, and further wherein the one or more sets of data are collected by a user device. The one or more sets of data are received from the user device. One or more criticalities are detected, where the one or more criticalities are detected by an artificial intelligence engine based on the one or more sets of data and a global knowledge base. One or more suggestions are determined, where the one or more suggestions are determined by the artificial intelligence engine based on the one or more criticalities and the global knowledge base. The one or more suggestions are sent to the user device.

### BRIEF DESCRIPTION OF THE DRAWINGS

FIG. 1 is a functional block diagram illustrating a biomedical wearable device environment, in accordance with an embodiment of the present invention.

FIG. 2 is an example of a smart joint monitor, in accordance with an embodiment of the present invention.

FIG. 3 is one possible block diagram of the function of the joint status monitor program, in accordance with an embodiment of the present invention.

FIG. 4 is a flowchart diagram depicting operational steps for the joint status monitor program for smart joint monitoring for bleeding disorder patients, on the distributed data processing environment of FIG. 1, in accordance with an embodiment of the present invention.

FIG. 5 depicts a block diagram of components of the computing device executing the joint status monitor program within the distributed data processing environment of FIG. 1, in accordance with an embodiment of the present invention.

### DETAILED DESCRIPTION

Recent discoveries in the era of Artificial Intelligence (AI) and the Internet of Things (IoT) has led to a convergence of medical science and technology drastically reducing physical hindrances. Among the deadliest and most frequently discussed congenital disorders, hemophilia, is still not curable among the masses due to the extremely high cost of the only available cure, gene therapy. This disorder is caused by the inability of the human liver to produce one of the essential the clotting factor proteins needed to form a blood clot to stop internal bleeding. Patients with hemophilia can hemorrhage internally anywhere in the body, but most commonly in the skeletal joints. Due to recurrent hemorrhages, a joint gradually becomes weaker and acts as a target joint, behaving as a weak link. If the target joint is not treated properly at a young age, it becomes deformed and degenerated over time, introducing disability into the joint. To avoid such a scenario, physicians strongly advise caring for the joints during the childhood of the patient. Since it is quite difficult to recognize the symptoms of hemorrhaging in a joint in a child, children with hemophilia often permanently damage one or more joints. This leads to reduced mobility, pain, and overall reduced quality of life.

The present invention is a method, computer program product, and system for smart joint monitoring for bleeding disorder patients. The present invention introduces a smart joint monitor for hemophiliac patients which monitors for vital symptoms that indicate damage occurring in a joint complex, analyzes the data from the smart joint monitor, and provides warnings to the user. The present invention provides both therapeutic and preventive recommendations. These recommendations are helpful for the user, as well as for the treating physician.

In an embodiment, the present invention consists of three essential stages, the symptom monitoring phase, the criticality detection phase, and the suggestion phase. In the symptom monitoring phase, the joint of the hemophiliac patient is monitored for the most predominant behavioral symptoms for detecting internal bleeding in the joint. In the criticality detection phase, after monitoring the intrinsic symptoms of an affected joint, the criticality of the joint and challenges commonly faced by the patient are detected. In the suggestion phase, the user is provided with vital suggestions on the immediate attention required to address the short-term crisis of the detected hemorrhage, as well as for long-term treatment and long-term improvement.

In an embodiment, the symptoms the present invention monitors include, but are not limited to, the following:

Joint temperature trend—a temperature increase of a specific joint is one of the predominant symptoms of an internal bleed. In addition, overstressing of a joint can be identified by monitoring for minute temperature alterations. In an embodiment, the present invention exploits existing fiber-optic sensors based on Fiber Bragg Grating (FBG), which reflects a particular wavelength, transmitting others. An FBG couple is insulated inside the polymer coating and implanted on the thread.

Synovium fluid or blood accumulation inside joint—synovium acts as a wrapper around skeletal joints and provides nourishment to the cartilage. Frequent bleeds into the joint of a hemophiliac causes blood to enter inside the synovium and creates a viscous substance inside the joint which makes the joint weaker and causes swelling, restricting natural movement. Identifying the initiation of fluid accumulation can prevent a severe joint bleed. In an embodiment, the present invention uses a group of existing soft strain sensors created with the composition of three types of sensors, namely a conductive elastomer cord sensor, a capacitive textile sensor and a cover-stitched sensor, to detect swelling around a joint. In an embodiment, these sensors communicate among themselves and gather data using a microcontroller.

Restriction in range of motion—common joint movements like flexion, extension, abduction, and adduction can be tracked by a Range of Motion (ROM) sensor. A rapid decrease in the range of motion can indicate a new or ongoing joint bleed. In the case of hemophiliac arthropathy, which is permanent joint disease occurring as a long-term consequence of repeated joint hemorrhaging, the ROM of the joint can also determine the degree of joint damage. In an embodiment, the present invention uses existing e-textile electro-goniometers for a precise measurement of the angles between connected body segments by measuring the flexion-extension angle. The goniometer is designed by coupling two layers of knitted piezo-resistive textures (KPF) within an insulating layer.

Presence of joint crepitus—joint crepitus is described as the popping, clicking or crackling sound from a joint. A long term effect of recurrent bleeding into a joint of a hemophiliac is damage to the synovium, which causes deterioration of the health of the joint cartilage by failing to provide sufficient nourishment. Since this cartilage works as a cushion or shock absorber for the joint, deteriorated or rough cartilage fails to stop the bones from rubbing together in the joint. Since cartilage can never regrow, this leads to joint crepitus of varying degrees. By detecting joint crepitus in its early phase and taking appropriate action, the present invention helps to avoid osteoarthritis in the joint. In an embodiment, the present invention measures joint sounds by an existing technique with a combination of Micro-Electro-Mechanical Systems (MEMS) microphones to sense sound pressure levels in the air and contact microphones to measure the vibrations at the surface of the skin. The present invention exploits AI to perform a quantitative and comparative study on these sounds and analyzes the state of the joint.

Joint movement, body posture and load balancing—by taking early intervention, it is possible to stop the joint health from degrading by notifying a physician when intensive activity or high acceleration and stress on a joint is incurred by the patient. Often it is difficult to determine the upper threshold of the activity level of the patient on the joint, which can lead to overstressing the joint and possibly severe joint bleeding. In an embodiment, the present invention compares movement patterns of a joint with the other bilateral joint, which is not equally affected, e.g., the affected left knee and the unaffected right knee, to identify a specific issue with the affected joint and take corrective actions. These corrective actions can include, for example, changes in posture or movements. In an embodiment, the present invention reads important musculoskeletal-related parameters to continuously monitor the joint movements during normal daily activities. With the help of intensity modulation or optical navigation methods in the optical sensor-based joint monitoring system, the present invention detects the precise movements of a joint. In another embodiment, textile-based strain sensors identify angle, motion, and rotation by monitoring the change in resistance.

In some cases, two bilateral limbs can behave differently due to recurrent internal hemorrhaging in one of them. This symptom occurs mostly in lower limbs of hemophiliac patients, where one joint becomes affected due to damage to another affected joint. For example, a patient whose right knee is injured due to hemorrhaging may tend to put too much body weight on the left knee to compensate for discomfort in the damaged right knee. This eventually leads to damage in the left knee due to the constant application of excessive weight to that knee.

In an embodiment, all the sensor data is captured locally in the smart joint monitor and is read by the software. In another embodiment, the sensor data is captured locally by a mobile device, e.g., a smart phone, and the software receives the sensor data from the mobile device. AI engines are applied to the data set, perform regression, and compare the data set with a data set from a global knowledge base to detect the criticality of the specific joint.

In an embodiment, the joint status monitor program develops the global knowledge base using a two-step strategy. First, when a hemophiliac patient visits a physician, the physician performs a thorough examination including running a series of tests, and either prescribes a treatment plan or updates the existing treatment plan based on the results of the examination and the tests. In an embodiment, the real-time data from the medical record and the treatment plan are manually stored in the global knowledge base. In an embodiment, this is the foundation of the knowledge base. In an embodiment, once patients start using the present invention, the set of sensors mentioned in this invention start collecting data, the data is processed, and finally recommendations are provided. In an embodiment, the recommendations are verified by a physician who can update or modify the recommendations if necessary. In an embodiment, the physician updates or modifies the recommendations, then the present invention adds the updated or modified recommendations to the global knowledge base. In an embodiment, the global knowledge base starts accumulating data and grows as the invention is used. In an embodiment, once the global knowledge base becomes sufficiently mature, and it has been reviewed and approved by subject matter experts, e.g., physicians, it is deployed to the real-time systems. In an embodiment, once deployed, the global knowledge base does not capture data automatically. In an embodiment, only subject matter experts, such as physicians, can add more data to the knowledge base based on authorized feedback received.

In an embodiment, if a new internal hemorrhage is detected, the present invention detects the degree of hemorrhaging and provides the user with a guideline on the quantity of Antihemophilic factor (AHF) required or, in the case of excessive blood loss, recommends a blood transfusion.

In an embodiment, in the case of a long term deformation, the present invention detects the stage of osteoarthritis that has occurred due to frequent hemorrhages. As a result of joint and muscle weakness, some musculoskeletal disorders can affect the joint. The degree of musculoskeletal disorders can be detected from the sensor data set and corrective actions can be taken by providing correction systems (e.g., knee brace, posture correction, etc.) to the patent.

In an embodiment, the AI algorithm of the present invention also suggests the treatment method depending on the joint condition. In an embodiment, if the joint is healthy enough to recover by corrective actions, then the present invention will suggest corrective physiotherapies with the help of a trained data set from the knowledge base. If the joint is beyond recovery by physiotherapy, the present invention suggests surgery to be performed for joint improvement and an increase in the lifestyle standard.

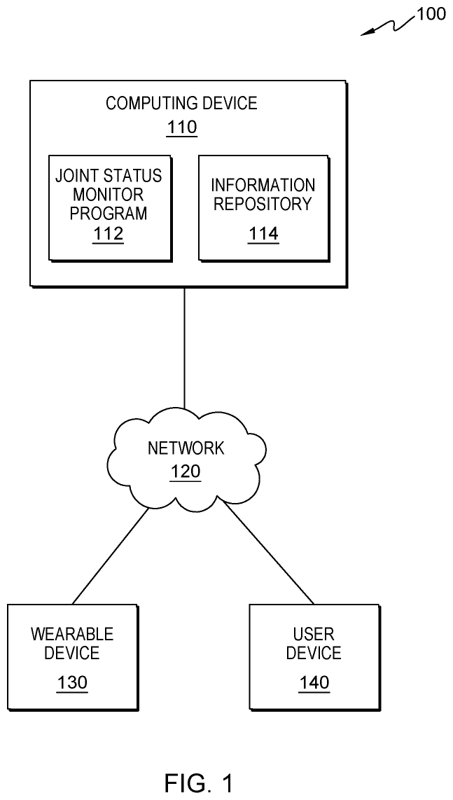

FIG. 1 is a functional block diagram illustrating a biomedical wearable device environment 100, suitable for operation of joint status monitor program 112 in accordance with at least one embodiment of the present invention. The term “distributed” as used herein describes a computer system that includes multiple, physically distinct devices that operate together as a single computer system. FIG. 1 provides only an illustration of one implementation and does not imply any limitations with regard to the environments in which different embodiments may be implemented. Many modifications to the depicted environment may be made by those skilled in the art without departing from the scope of the invention as recited by the claims.

Biomedical wearable device environment 100 includes computing device 110, wearable device 130, and user device 140, all connected to network 120. Network 120 can be, for example, a telecommunications network, a local area network (LAN), a wide area network (WAN), such as the Internet, or a combination of the three, and can include wired, wireless, or fiber optic connections. Network 120 can include one or more wired and/or wireless networks that are capable of receiving and transmitting data, voice, and/or video signals, including multimedia signals that include voice, data, and video information. In general, network 120 can be any combination of connections and protocols that will support communications between computing device 110, wearable device 130, user device 140, and other computing devices (not shown) within biomedical wearable device environment 100.

Computing device 110 can be a standalone computing device, a management server, a web server, a mobile computing device, or any other electronic device or computing system capable of receiving, sending, and processing data. In an embodiment, computing device 110 can be a laptop computer, a tablet computer, a netbook computer, a personal computer (PC), a desktop computer, a personal digital assistant (PDA), a smart phone, or any programmable electronic device capable of communicating with wearable device 130, user device 140, and other computing devices (not shown) within biomedical wearable device environment 100 via network 120. In another embodiment, computing device 110 can represent a server computing system utilizing multiple computers as a server system, such as in a cloud computing environment. In yet another embodiment, computing device 110 represents a computing system utilizing clustered computers and components (e.g., database server computers, application server computers) that act as a single pool of seamless resources when accessed within biomedical wearable device environment 100.

In an embodiment, computing device 110 includes joint status monitor program 112. In an embodiment, joint status monitor program 112 is a program, application, or subprogram of a larger program for smart joint monitoring for bleeding disorder patients. In an alternative embodiment, joint status monitor program 112 may be located on any other device accessible by computing device 110 via network 120.

In an embodiment, computing device 110 includes information repository 114. In an embodiment, information repository 114 may be managed by joint status monitor program 112. In an alternate embodiment, information repository 114 may be managed by the operating system of the device, alone, or together with, joint status monitor program 112. Information repository 114 is a data repository that can store, gather, compare, and/or combine information. In some embodiments, information repository 114 is located externally to computing device 110 and accessed through a communication network, such as network 120. In some embodiments, information repository 114 is stored on computing device 110. In some embodiments, information repository 114 may reside on another computing device (not shown), provided that information repository 114 is accessible by computing device 110. Information repository 114 includes, but is not limited to, sensor data, knowledgebase data, user data, system configuration data, and other data that is received by joint status monitor program 112 from one or more sources, and data that is created by joint status monitor program 112.

Information repository 114 may be implemented using any volatile or non-volatile storage media for storing information, as known in the art. For example, information repository 114 may be implemented with a tape library, optical library, one or more independent hard disk drives, multiple hard disk drives in a redundant array of independent disks (RAID), solid-state drives (SSD), or random-access memory (RAM). Similarly, the information repository 114 may be implemented with any suitable storage architecture known in the art, such as a relational database, an object-oriented database, or one or more tables.

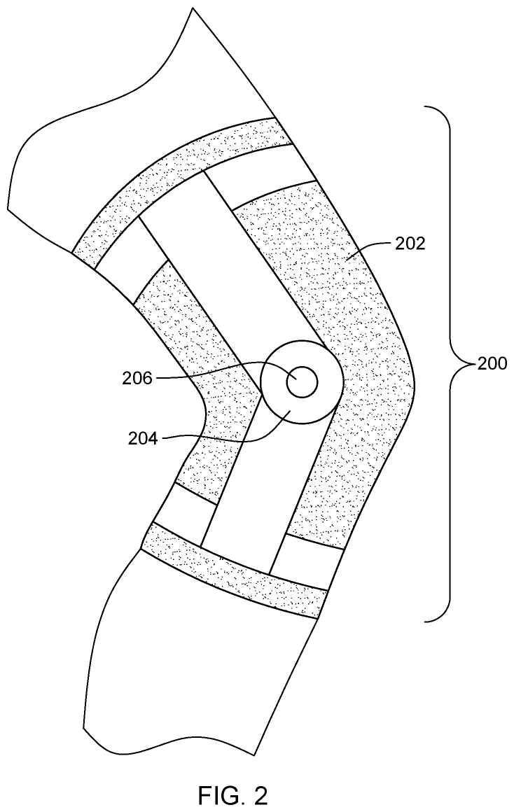

In an embodiment, wearable device 130 is a smart joint monitor that is worn on a joint on the user's body, e.g., a knee or ankle, with a plurality of sensors to gather data on the monitored joint. In an embodiment, wearable device 130 collects data from the plurality of sensors, which is then read by joint status monitor program 112. In an embodiment, wearable device 130 connects to user device 140 over network 120, which reads the monitored data from wearable device 130, which is read in turn from user device 140 by joint status monitor program 112. An example of wearable device 130 is shown in FIG. 2 and described below.

User device 140 can be a can be a smart phone, standalone computing device, a mobile computing device, or any other electronic device or computing system that includes the ability to receive, send, and process data. In an embodiment, user device 140 can be a smart phone, a laptop computer, a tablet computer, a netbook computer, a personal computer (PC), a desktop computer, a personal digital assistant (PDA), or any programmable electronic device that is capable of communicating with other computing devices (not shown) within biomedical wearable device environment 100 via network 120. In an embodiment, user device 140 collects sensor data from wearable device 130 over network 120. In an embodiment, user device 140 connects to wearable device via a personnel network, e.g., Bluetooth®.

FIG. 2 is an example of a smart joint monitor, e.g., wearable device 130 from FIG. 1, that is worn by a user, in accordance with an embodiment of the present invention. The example of FIG. 2 illustrates a smart joint monitor for a knee joint. In this example, smart joint monitor 200 includes composite smart textile 202, which represents one possible sensor for smart joint monitor 200. In an embodiment, the smart textile sensor can monitor movement in the joint. In another embodiment, the smart textile sensor can monitor precise angular movement in the joint. In yet another embodiment, the smart textile sensor can monitor fluid or blood accumulation in the joint. In this example, smart joint monitor 200 also includes textile goniometer 204. A goniometer is an instrument for the precise measurement of angles. In an embodiment, the textile goniometer can monitor precise angular movement in the joint. In this example, smart joint monitor 200 also includes embedded microphone 206. In an embodiment, the microphone is embedded in smart joint monitor 200 to detect crepitus, which is the noise generated by bone-on-bone grinding due to loss of lubricating cartilage in the joint.

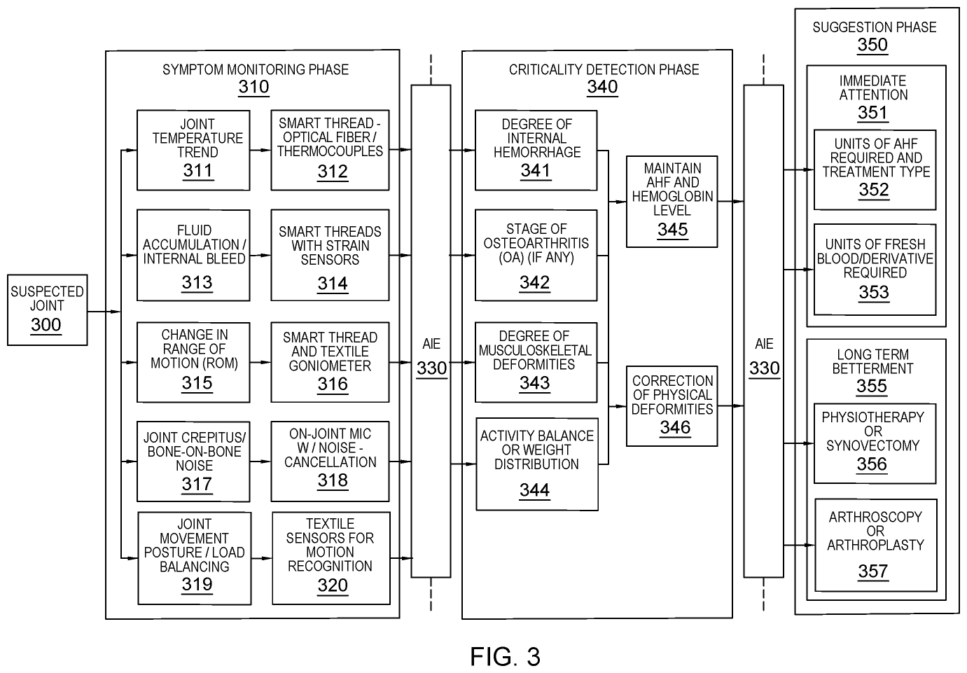

FIG. 3 is one possible block diagram of the function of joint status monitor program 112, in accordance with an embodiment of the present invention. FIG. 3 includes suspected joint 300, which is the actual joint to be monitored by joint status monitor program 112. FIG. 3 illustrates the three essential stages of joint status monitor program 112, symptom monitoring phase 310, criticality detection phase 340, and suggestion phase 350.

In an embodiment, symptom monitoring phase 310 is the phase of joint status monitor program 112 where the program monitors suspected joint 300 and collects data for joint status monitor program 112. In an embodiment, symptom monitoring phase 310 includes, but is not limited to, the following symptoms: joint temperature trend 311, fluid accumulation/internal bleed 313, change in Range of Motion (ROM) 315, Joint crepitus/bone-on-bone noise 317, and joint movement posture/load balancing 319. The use of these symptoms are described above. These symptoms are monitored, respectively, by the following sensors: smart thread—optical fiber thermocouples 312, smart threads with strain sensors 314, smart thread with textile goniometer 316, on-joint microphone with noise cancellation 318, and textile sensors for motion recognition 320.

In an embodiment, symptom monitoring phase 310 receives the data from these sensors and inputs that data into Artificial Intelligence Engine (AIE) 330. In an embodiment, this is the AI engine that is applied to the data set, performs regression analysis on the data set, compares the data set with a data set from the global knowledge base, and detects the criticality of the specific joint.

In an embodiment, criticality detection phase 340 is the phase of joint status monitor program 112 where the AI engine detects the criticality of the joint and the common challenges faced by the patient, based on the output of AIE 330. In an embodiment, criticality detection phase 340 includes, but is not limited to, the following critical conditions: degree of internal hemorrhage 341, stage of osteoarthritis (OA) 342, degree of musculoskeletal deformities 343, and activity balance or weight distribution 344. These conditions are described above. These conditions generate the following proposed solutions: maintain AHF and hemoglobin level 345 and correction of physical deformities 346. In an embodiment, symptom monitoring phase 310 inputs the proposed solutions into AIE 330.

In an embodiment, suggestion phase 350 provides vital suggestions on immediate attention required to address the short term situation and also aims for long term betterment, based on the output from AIE 330. In an embodiment, suggestion phase 350 includes, but is not limited to, the following suggestions: immediate attention 351 (including units of AHF required and treatment type 352 and units of fresh blood or blood derivative required 353) and long term betterment 355 (including physiotherapy or synovectomy 356 and arthroscopy or arthroplasty 357). Synovectomy is a procedure where the synovial tissue surrounding a joint is removed. Arthroscopy and arthroplasty are types of surgery. These suggestions are merely examples of the possible suggestions that various embodiments of the present invention could make.

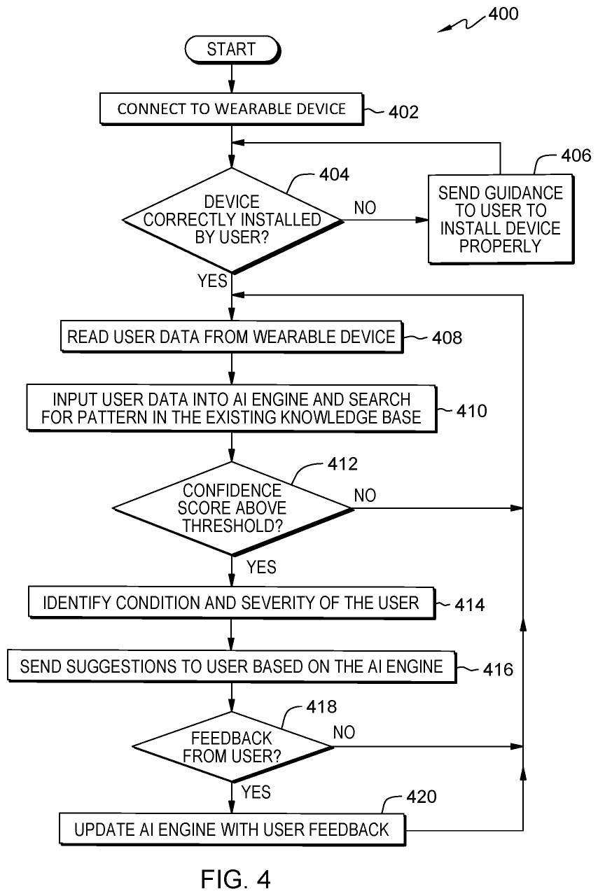

FIG. 4 is a flow chart diagram of workflow 400 depicting operational steps for joint status monitor program 112 for smart joint monitoring for bleeding disorder patients. In an alternative embodiment, the steps of workflow 400 may be performed by any other program while working with joint status monitor program 112. In an embodiment, joint status monitor program 112 connects to wearable device 130, e.g., smart joint monitor 200 from FIG. 2, to begin monitoring the joint. In an embodiment, joint status monitor program 112 determines if wearable device 130 was properly positioned by the user. In an embodiment, joint status monitor program 112 sends guidance to the user to help the user properly position wearable device 130. In an embodiment, joint status monitor program 112 reads data from wearable device 130. In an embodiment, joint status monitor program 112 inputs the data into the AI engine and searches the global knowledge base to identify the condition. In an embodiment, joint status monitor program 112 determines if the confidence score for the possible match found in the previous step exceeds a threshold. In an embodiment, joint status monitor program 112 identifies the condition of the user and the severity of the condition based on the results from the AI engine. In an embodiment, joint status monitor program 112 sends appropriate suggestions to the user based on the results of the AI engine. In an embodiment, joint status monitor program 112 determines if the user has provided any feedback. In an embodiment, joint status monitor program 112 updates the global knowledge base with the feedback from the user.

It should be appreciated that embodiments of the present invention provide at least for smart joint monitoring for bleeding disorder patients. However, FIG. 4 provides only an illustration of one implementation and does not imply any limitations with regard to the environments in which different embodiments may be implemented. Many modifications to the depicted environment may be made by those skilled in the art without departing from the scope of the invention as recited by the claims.

Joint status monitor program 112 connects to the wearable device (step 402). In an embodiment, joint status monitor program 112 connects to wearable device 130, e.g., smart joint monitor 200 from FIG. 2, to begin monitoring the joint. In an embodiment, joint status monitor program 112 connects to wearable device 130 via network 120. In another embodiment, joint status monitor program 112 connects to wearable device 130 via user device 140. In another embodiment, joint status monitor program 112 connects to wearable device 130 via user device 140 using a personal network, e.g., Bluetooth.

Joint status monitor program 112 determines if the wearable device is correctly positioned by the user (decision block 404). In an embodiment, joint status monitor program 112 determines if wearable device 130 was properly positioned by the user. In an embodiment, joint status monitor program 112 determines if wearable device 130 was properly positioned by the user by analyzing data received from the smart joint monitor. In an embodiment, data received from the smart joint monitor is within an acceptable range, then joint status monitor program 112 determines that wearable device 130 was properly positioned by the user. In an embodiment, the acceptable range is a system default. In another embodiment, the acceptable range is input by a subject matter expert, e.g., a physician. In an embodiment, joint status monitor program 112 determines if the smart joint monitor was properly positioned by the user by receiving images of the proper positioning of the smart joint monitor from the user. In an embodiment, these images are received from user device 140 from FIG. 1. In an embodiment, if joint status monitor program 112 determines that the smart joint monitor was properly positioned by the user (“yes” branch, decision block 404), then joint status monitor program 112 proceeds to step 408. In an embodiment, if joint status monitor program 112 determines that the smart joint monitor was not properly positioned by the user (“no” branch, decision block 404), then joint status monitor program 112 proceeds to step 406.

Joint status monitor program 112 sends guidance to the user device to position the wearable device properly (step 406). In an embodiment, joint status monitor program 112 sends guidance to the user to help the user properly position wearable device 130. In an embodiment, joint status monitor program 112 sends guidance to the user via user device 140. In an embodiment, joint status monitor program 112 sends instructional videos to the user to position the monitor properly. In another embodiment, joint status monitor program 112 sends text instructions to the user. In an embodiment, joint status monitor program 112 sends instructions to the user in any manner as would be known to a person having skill in the art.

Joint status monitor program 112 reads user data from the wearable device (step 408). In an embodiment, joint status monitor program 112 reads data from wearable device 130. In an embodiment, joint status monitor program 112 reads the data continuously. In an embodiment, user device 140 interfaces with wearable device 130 to collect the data, and joint status monitor program 112 receives the data from user device 140. In an embodiment, the user data includes all data collected from any of the sensors on wearable device 130, such as those described in the examples of FIG. 2 and FIG. 3 above.

Joint status monitor program 112 inputs user data into the AI engine and searches for a pattern in the existing knowledge base (step 410). In an embodiment, joint status monitor program 112 inputs the data into the AI engine and searches the global knowledge base to identify the condition. In an embodiment, if joint status monitor program 112 finds a possible match for the pattern in the data set, then joint status monitor program 112 determines a confidence score for the possible match based on the results from the AI engine.

In an embodiment, joint status monitor program 112 pre-processes the sensor data to generate more relevant features. In an embodiment, some of the relevant features generated from the raw data include, but are not limited to, the following: rate of change of temperature, i.e., monthly, weekly, daily, standard deviation; timings of fluid accumulation or internal bleedings, i.e., mean of the time difference between consecutive bleedings; bone to bone noise, time length of the noise, and noise frequency and intensity; rate of change of range or motion, i.e., monthly, weekly, daily, standard deviation; and joint motion, posture, load and the timing of each.

In an embodiment, once the above relevant features are generated from the data points, joint status monitor program 112 uses classification algorithms for multi-class classification to provide the output for each one of the recommendation scenarios. In an embodiment, a composite recommendation is provided based on each output model. In an embodiment, the recommendation consists of a combination of at least, but is not limited to, the following details based on the current scenario: physiotherapy or synovectomy; arthroscopy and arthroplasty; AHF requirement; medication for Hemophilia B; and a blood transfusion. In an embodiment, after joint status monitor program 112 provides the recommendation and an action is taken, that action becomes an input to the future recommendation for that specific user.

In an embodiment, since this is a multi-class classification problem, a confidence score is determined for each class. In an embodiment, an output class is eligible for recommendation when the confidence score for that class exceeds a pre-determined threshold. In an embodiment, the confidence score is determined during the model training phase. In an embodiment, the confidence score can be fine-tuned by a subject matter expert, e.g., a physician.

Joint status monitor program 112 determines if the confidence score is above a threshold (decision block 412). In an embodiment, joint status monitor program 112 determines if the confidence score for the possible match found in step 410 exceeds a threshold. In an embodiment, the threshold is a system default. In another embodiment, the threshold is received from the user, e.g., a value determined by the user's physician based on the particular condition of the user. In an embodiment, if joint status monitor program 112 determines that the confidence score exceeds the threshold (“yes” branch, decision block 412), then joint status monitor program 112 proceeds to step 414. In an embodiment, if joint status monitor program 112 determines that the confidence score does not exceed the threshold (“no” branch, decision block 412), then joint status monitor program 112 returns to step 408 to continue to collect data from wearable device 130.

Joint status monitor program 112 identifies the condition and severity of the user (step 414). In an embodiment, joint status monitor program 112 identifies the condition of the user and the severity of the condition based on the results from the AI engine in step 410. The health condition of a hemophiliac patient may fluctuate in many ways, if proper treatment is not provided at the appropriate time. In an embodiment, joint status monitor program 112 uses the AI engine to process parameters read from the sensors. This creates an accumulated result which depicts the condition of the user and also gives clarity about the severity of the condition. The present invention uses the data from the multiple sensors because monitoring only one parameter may not provide sufficient information about the condition of the user which can lead to overlooking a severe condition where prompt treatment measures are required. These kinds of misinterpretation often lead to health condition deterioration for the patients. For example joint pain may be caused by acute osteoarthritis in the joint or may be the result of an active hemorrhage. Blood accumulation around the synovial fluid and reduction in ROM will confirm an acute hemorrhage in the joint and requires performing immediate factor replacement therapy.

Joint status monitor program 112 sends suggestions to user based on the AI engine (step 416). In an embodiment, joint status monitor program 112 sends appropriate suggestions to the user based on the results of the AI engine from step 410. For example, the suggestions of suggestion phase 350 from FIG. 3 are sent by joint status monitor program 112 if appropriate.

In an embodiment, after joint status monitor program 112 uses the AI engine to process the data gathered by the multiple sensors, the recommendation system of joint status monitor program 112 determines two sets of recommendations depending on the time frame during which they are going to be applied. In an embodiment, the first set of recommendations are those remedies that demand immediate attention. This can be considered as a lifesaving measures where failing to provide a timely resolution may cause a serious injury or loss of life of a patient. In an embodiment, the artificial intelligence model is trained to prioritize those normalized parameters that lead to this set of remedies which demand immediate application. In an embodiment, the second set recommendations deal with long-term betterment of the patient, where the focus is on increasing the lifestyle standard of the hemophiliac patient. In an embodiment, these recommendations may be loosely bound and can be manipulated to a small degree by the machine learning engine for the purpose of fine-tuning to the specific data gathered in step 408.

Joint status monitor program 112 determines if there is feedback from user (decision block 418). In an embodiment, joint status monitor program 112 determines if the user has provided any feedback. In an embodiment, if joint status monitor program 112 determines that the user has provided any feedback (“yes” branch, decision block 418), then joint status monitor program 112 proceeds to step 420. In an embodiment, if joint status monitor program 112 determines that the user has not provided any feedback (“no” branch, decision block 418), then joint status monitor program 112 returns to step 408 to continue to collect data from wearable device 130.

Joint status monitor program 112 updates the AI engine with user feedback (step 420). In an embodiment, joint status monitor program 112 updates the global knowledge base with the feedback from the user. Joint status monitor program 112 then returns to step 408 to continue to collect data from wearable device 130.

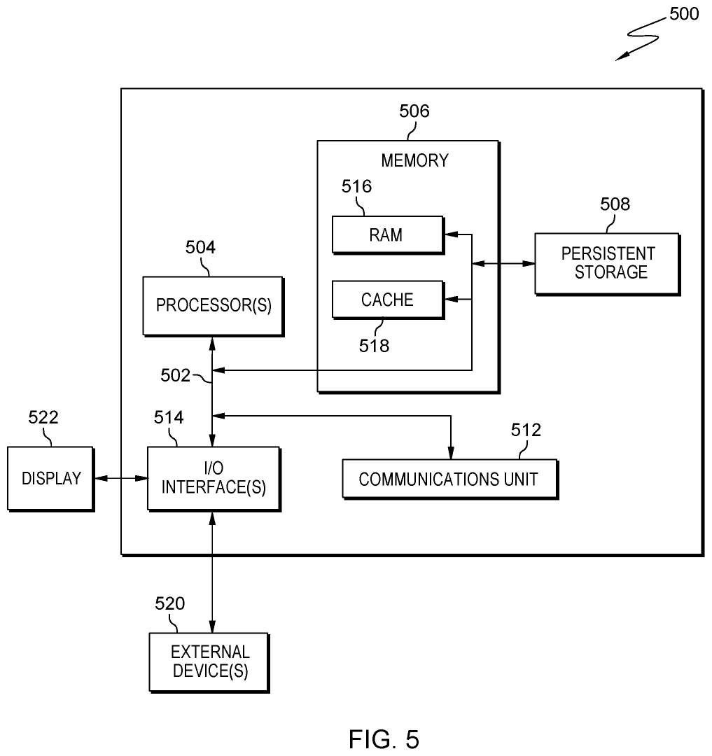

FIG. 5 is a block diagram depicting components of computing device 110 suitable for joint status monitor program 112, in accordance with at least one embodiment of the invention. FIG. 5 displays computer 500; one or more processor(s) 504 (including one or more computer processors); communications fabric 502; memory 506, including random-access memory (RAM) 516 and cache 518; persistent storage 508; communications unit 512; I/O interfaces 514; display 522; and external devices 520. It should be appreciated that FIG. 5 provides only an illustration of one embodiment and does not imply any limitations with regard to the environments in which different embodiments may be implemented. Many modifications to the depicted environment may be made.

As depicted, computer 500 operates over communications fabric 502, which provides communications between computer processor(s) 504, memory 506, persistent storage 508, communications unit 512, and I/O interface(s) 514. Communications fabric 502 may be implemented with any architecture suitable for passing data or control information between processors 504 (e.g., microprocessors, communications processors, and network processors), memory 506, external devices 520, and any other hardware components within a system. For example, communications fabric 502 may be implemented with one or more buses.

Memory 506 and persistent storage 508 are computer readable storage media. In the depicted embodiment, memory 506 comprises RAM 516 and cache 518. In general, memory 506 can include any suitable volatile or non-volatile computer readable storage media. Cache 518 is a fast memory that enhances the performance of processor(s) 504 by holding recently accessed data, and near recently accessed data, from RAM 516.

Program instructions for joint status monitor program 112 may be stored in persistent storage 508, or more generally, any computer readable storage media, for execution by one or more of the respective computer processors 504 via one or more memories of memory 506. Persistent storage 508 may be a magnetic hard disk drive, a solid-state disk drive, a semiconductor storage device, read only memory (ROM), electronically erasable programmable read-only memory (EEPROM), flash memory, or any other computer readable storage media that is capable of storing program instruction or digital information.

The media used by persistent storage 508 may also be removable. For example, a removable hard drive may be used for persistent storage 508. Other examples include optical and magnetic disks, thumb drives, and smart cards that are inserted into a drive for transfer onto another computer readable storage medium that is also part of persistent storage 508.

Communications unit 512, in these examples, provides for communications with other data processing systems or devices. In these examples, communications unit 512 includes one or more network interface cards. Communications unit 512 may provide communications through the use of either or both physical and wireless communications links. In the context of some embodiments of the present invention, the source of the various input data may be physically remote to computer 500 such that the input data may be received, and the output similarly transmitted via communications unit 512.

I/O interface(s) 514 allows for input and output of data with other devices that may be connected to computer 500. For example, I/O interface(s) 514 may provide a connection to external device(s) 520 such as a keyboard, a keypad, a touch screen, a microphone, a digital camera, and/or some other suitable input device. External device(s) 520 can also include portable computer readable storage media such as, for example, thumb drives, portable optical or magnetic disks, and memory cards. Software and data used to practice embodiments of the present invention, e.g., joint status monitor program 112, can be stored on such portable computer readable storage media and can be loaded onto persistent storage 508 via I/O interface(s) 514. I/O interface(s) 514 also connect to display 522.

Display 522 provides a mechanism to display data to a user and may be, for example, a computer monitor. Display 522 can also function as a touchscreen, such as a display of a tablet computer.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product. The computer program product may include a computer readable storage medium (or media) having computer readable program instructions thereon for causing a processor to carry out aspects of the present invention.

The computer readable storage medium can be any tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method for monitoring skeletal joints in patients with bleeding disorders, the computer-implemented method comprising:
receiving, by one of more computer processors, one or more sets of data from a wearable device, wherein the wearable device is a smart joint monitor that includes one or more sensors;
detecting, by the one or more computer processors, one or more criticalities, wherein the one or more criticalities are detected by an artificial intelligence engine based on the one or more sets of data and a global knowledge base; and
determining, by the one or more computer processors, one or more suggestions, wherein the one or more suggestions are determined by the artificial intelligence engine based on the one or more criticalities and the global knowledge base.

2. The computer-implemented method of claim 1, wherein detecting the one or more criticalities, wherein the one or more criticalities are detected by the artificial intelligence engine based on the one or more sets of data and the global knowledge base comprises:
applying, by the one or more computer processors, the one or more sets of data to the artificial intelligence engine;
performing, by the one or more computer processors, a regression analysis on the one or more sets of data; and
comparing, by the one or more computer processors, a results of the regression analysis to the global knowledge base to detect the one or more criticalities.

3. The computer-implemented method of claim 1, wherein the one or more sensors include at least one of a smart thread thermocouple, an optical fiber thermocouple, one or more smart threads with strain sensors, a smart thread with textile goniometer, an on-joint microphone with noise cancellation, a micro-electro-mechanical systems (MEMS) microphone, and one or more textile sensors for motion recognition.

4. The computer-implemented method of claim 1, wherein the one or more sets of data include at least one of a joint temperature, a fluid accumulation, a change in range of motion (ROM), a joint crepitus, a joint movement, a posture, and a joint load balancing.

5. The computer-implemented method of claim 1, wherein the one or more criticalities include at least one of a degree of internal hemorrhaging, a stage of osteoarthritis, a degree of musculoskeletal deformation, an activity balance, and a weight distribution.

6. The computer-implemented method of claim 1, wherein the one or more suggestions include at least one of a number of units of Antihemophilic factor recommended and a treatment type recommended, a number of units of fresh blood recommended, a number of units of blood derivative recommended, a physiotherapy recommended, a synovectomy recommended, an arthroscopy recommended, and an arthroplasty recommended.

7. A computer program product for monitoring skeletal joints in patients with bleeding disorders, the computer program product comprising one or more computer readable storage media and program instructions stored on the one or more computer readable storage media, the program instructions including instructions to:
receive one or more sets of data from a wearable device, wherein the wearable device is a smart joint monitor that includes one or more sensors;
detect one or more criticalities, wherein the one or more criticalities are detected by an artificial intelligence engine based on the one or more sets of data and a global knowledge base; and
determine one or more suggestions, wherein the one or more suggestions are determined by the artificial intelligence engine based on the one or more criticalities and the global knowledge base.

8. The computer program product of claim 7, wherein detecting the one or more criticalities, wherein the one or more criticalities are detected by the artificial intelligence engine based on the one or more sets of data and the global knowledge base comprises one or more of the following program instructions, stored on the one or more computer readable storage media, to:
apply the one or more sets of data to the artificial intelligence engine;
perform a regression analysis on the one or more sets of data; and
compare a results of the regression analysis to the global knowledge base to detect the one or more criticalities.

9. The computer program product of claim 7, wherein the one or more sensors include at least one of a smart thread thermocouple, an optical fiber thermocouple, one or more smart threads with strain sensors, a smart thread with textile goniometer, an on-joint microphone with noise cancellation, a micro-electro-mechanical systems (MEMS) microphone, and one or more textile sensors for motion recognition.

10. The computer program product of claim 7, wherein the one or more sets of data include at least one of a joint temperature, a fluid accumulation, a change in range of motion (ROM), a joint crepitus, a joint movement, a posture, and a joint load balancing.

11. The computer program product of claim 7, wherein the one or more criticalities include at least one of a degree of internal hemorrhaging, a stage of osteoarthritis, a degree of musculoskeletal deformation, an activity balance, and a weight distribution.

12. The computer program product of claim 7, wherein the one or more suggestions include at least one of a number of units of Antihemophilic factor recommended and a treatment type recommended, a number of units of fresh blood recommended, a number of units of blood derivative recommended, a physiotherapy recommended, a synovectomy recommended, an arthroscopy recommended, and an arthroplasty recommended.

13. A computer system for monitoring skeletal joints in patients with bleeding disorders, the computer system comprising:
one or more computer processors;
one or more computer readable storage media; and
program instructions stored on the one or more computer readable storage media for execution by at least one of the one or more computer processors, the stored program instructions including instructions to:
receive one or more sets of data from a wearable device, wherein the wearable device is a smart joint monitor that includes one or more sensors;
detect one or more criticalities, wherein the one or more criticalities are detected by an artificial intelligence engine based on the one or more sets of data and a global knowledge base; and
determine one or more suggestions, wherein the one or more suggestions are determined by the artificial intelligence engine based on the one or more criticalities and the global knowledge base.

14. The computer system of claim 13, wherein detecting the one or more criticalities, wherein the one or more criticalities are detected by the artificial intelligence engine based on the one or more sets of data and the global knowledge base comprises one or more of the following program instructions, stored on the one or more computer readable storage media, to:
apply the one or more sets of data to the artificial intelligence engine;
perform a regression analysis on the one or more sets of data; and
compare a results of the regression analysis to the global knowledge base to detect the one or more criticalities.

15. The computer system of claim 13, wherein the one or more sensors include at least one of a smart thread thermocouple, an optical fiber thermocouple, one or more smart threads with strain sensors, a smart thread with textile goniometer, an on-joint microphone with noise cancellation, a micro-electro-mechanical systems (MEMS) microphone, and one or more textile sensors for motion recognition.

16. The computer system of claim 13, wherein the one or more sets of data include at least one of a joint temperature, a fluid accumulation, a change in range of motion (ROM), a joint crepitus, a joint movement, a posture, and a joint load balancing.

17. The computer system of claim 13, wherein the one or more criticalities include at least one of a degree of internal hemorrhaging, a stage of osteoarthritis, a degree of musculoskeletal deformation, an activity balance, and a weight distribution.

18. The computer system of claim 13, wherein the one or more suggestions include at least one of a number of units of Antihemophilic factor recommended and a treatment type recommended, a number of units of fresh blood recommended, a number of units of blood derivative recommended, a physiotherapy recommended, a synovectomy recommended, an arthroscopy recommended, and an arthroplasty recommended.

19. A computer-implemented method for monitoring skeletal joints in patients with bleeding disorders, the computer-implemented method comprising:
collecting, by one of more computer processors, one or more sets of data from a wearable device, wherein the wearable device is a smart joint monitor that includes one or more sensors, and further wherein the one or more sets of data are collected by a user device;
receiving, by one of more computer processors, the one or more sets of data from the user device;
detecting, by the one or more computer processors, one or more criticalities, wherein the one or more criticalities are detected by an artificial intelligence engine based on the one or more sets of data and a global knowledge base;
determining, by the one or more computer processors, one or more suggestions, wherein the one or more suggestions are determined by the artificial intelligence engine based on the one or more criticalities and the global knowledge base; and
sending, by the one or more computer processors, the one or more suggestions to the user device.

20. The computer-implemented method of claim 19, further comprising:
determining, by the one or more computer processors, whether the wearable device is properly positioned on a user;
responsive to determining that the wearable device is not properly positioned on the user, sending, by the one or more computer processors, one or more instructions to properly position the wearable device to the user device; and
displaying, by the one or more computer processors, the one or more instructions to properly position the wearable device on the user device.

21. The computer-implemented method of claim 19, wherein the one or more sensors include at least one of a smart thread thermocouple, an optical fiber thermocouple, one or more smart threads with strain sensors, a smart thread with textile goniometer, an on-joint microphone with noise cancellation, a micro-electro-mechanical systems (MEMS) microphone, and one or more textile sensors for motion recognition.

22. The computer-implemented method of claim 19, wherein the one or more criticalities include at least one of a degree of internal hemorrhaging, a stage of osteoarthritis, a degree of musculoskeletal deformation, an activity balance, and a weight distribution.

23. The computer-implemented method of claim 19, wherein the one or more suggestions include at least one of a number of units of Antihemophilic factor recommended and a treatment type recommended, a number of units of fresh blood recommended, a number of units of blood derivative recommended, a physiotherapy recommended, a synovectomy recommended, an arthroscopy recommended, and an arthroplasty recommended.

24. A computer program product for monitoring skeletal joints in patients with bleeding disorders, the computer program product comprising one or more computer readable storage media and program instructions stored on the one or more computer readable storage media, the program instructions including instructions to:
collect one or more sets of data from a wearable device, wherein the wearable device is a smart joint monitor that includes one or more sensors, and further wherein the one or more sets of data are collected by a user device;
receive the one or more sets of data from the user device;
detect one or more criticalities, wherein the one or more criticalities are detected by an artificial intelligence engine based on the one or more sets of data and a global knowledge base;
determine one or more suggestions, wherein the one or more suggestions are determined by the artificial intelligence engine based on the one or more criticalities and the global knowledge base; and
send the one or more suggestions to the user device.

25. The computer program product of claim 24, further comprising one or more of the following program instructions, stored on the one or more computer readable storage media, to:
determine whether the wearable device is properly positioned on a user;
responsive to determining that the wearable device is not properly positioned on the user, send one or more instructions to properly position the wearable device to the user device; and
display the one or more instructions to properly position the wearable device on the user device.
